(19) United States
(12) Patent Application Publication
(10) Pub. No.: US 2003/0022234 A1
(43) Pub. Date: Jan. 30, 2003

(54) Method and system to conduct a combinatorial high throughput screening experiment

(76) Inventor: James Norman Cawse, Pittsfield, MA (US)

Correspondence Address: GENERAL ELECTRIC COMPANY, GLOBAL RESEARCH CENTER, PATENT DOCKET RM. 4A59, PO BOX 8, BLDG. K-1 ROSS, NISKAYUNA, NY, 12309, US

(21) Appl. No.: 09/681,753

(22) Filed: May 31, 2001

(51) Int. Cl. G01N 33/53; G06F 19/00; G01N 33/48; G01N 33/50; G01N 33/543

(52) U.S. Cl. 435/7.1; 436/518; 702/19

(57) ABSTRACT

In a method, factors are selected for an experiment and interactions among levels of the factors are estimated. A probability value of positive interactions is then assigned for each of the estimated interactions. A combinatorial high throughput screening (CHTS) method is effected on an experimental space representing the levels and the probabilities for each interaction are adjusted according to results of the CHTS method. A system for conducting an experiment includes a reactor for effecting a CHTS method on an experimental space to produce results and a programmed controller that stores an assigned probability value for estimated positive interactions between levels of factors of the experimental space and adjusts the probabilities for each interaction according to results of the CHTS method.

### FEDERAL RESEARCH STATEMENT

This invention was made with government support under Contract No. 70NAN89H3038 awarded by NIST. The government may have certain rights to the invention.

### BACKGROUND OF INVENTION

The present invention relates to a method and system to conduct a combinatorial high throughput screening (CHTS) experiment.

Combinatorial organic synthesis (COS) is a high throughput screening (HTS) method that was developed for pharmaceuticals. COS uses systematic and repetitive synthesis to produce diverse molecular entities formed from sets of chemical “building blocks.” As with traditional research, COS relies on experimental synthesis methodology. However instead of synthesizing a single compound, COS exploits automation and miniaturization to produce large libraries of compounds through successive stages, each of which produces a chemical modification of an existing molecule of a preceding stage. Libraries are physical, trackable collections of samples resulting from a definable set of the COS process or reaction steps. The libraries comprise compounds that can be screened for various activities.

Combinatorial high throughput screening (CHTS) is an HTS method that incorporates characteristics of COS. The CHTS methodology is marked by the search for high order synergies and effects of complex combinations of experimental variables through the use of large arrays in which multiple factors can be varied through multiple levels. Factors of an experiment can be varied within an array (typically formulation variables) and between an array and a condition (both formulation and processing variables). Results from the CHTS experiment can be used to compare properties of the products in order to discover “leads” formulations and/or processing conditions that indicate commercial potential.

The steps of a CHTS methodology can be broken down into generic operations including selecting chemicals to be used in an experiment, introducing the chemicals into a formulation system (typically by weighing and dissolving to form stock solutions), combining aliquots of the solutions into formulations or mixtures in a geometrical array (typically by the use of a pipetting robot), processing the array of chemical combinations into products and evaluating the products to produce results.

Typically, CHTS methodology is characterized by parallel reactions at a micro scale. In one aspect, CHTS can be described as a method comprising (A) an iteration of steps of (i) selecting a set of reactants, (ii) reacting the set and (iii) evaluating a set of products of the reacting step and (B) repeating the iteration of steps (i), (ii) and (iii) wherein a successive set of reactants selected for a step (i) is chosen as a result of an evaluating step (iii) of a preceding iteration.

The study of catalyzed chemical reactions by CHTS involves the investigation of a complex experimental space characterized by multiple qualitative and quantitative factor levels. Typically, the interactions of a catalyzed chemical reaction such as a carbonylation reaction can involve interactions of an order of 6 or 9 or greater. An investigator must carefully set up a CHTS experiment in order to effectively examine such a complex space. Reactant identities and variables, process identities and variables and levels of combinations of factors, must be chosen to define a space that will provide meaningful results.

In most instances, an investigator conducts the CHTS experiment for the benefit of a client, who for example, may be a customer from outside the investigator”s company or co-worker from another department within the company. In any case, the client attempts to clearly articulate its expectations for the experiment to the investigator while at the same time, the investigator articulates capabilities and limitations of the CHTS methodology. It is difficult but critical to translate the articulations of the client and investigator into an experiment definition for the CHTS method. The complexity of a catalyzed chemical experimental space makes translation of needs and capabilities into an experiment definition even more difficult. There is a need for a method and system to conduct an experiment according to specific needs of a client and capabilities of the CHTS method.

### SUMMARY OF INVENTION

The invention meets this need by a providing a method and system to develop an experiment definition for a CHTS experiment. In the method, factors are selected for the experiment and interactions among levels of the factors are estimated. A probability value of positive interactions is then assigned for each of the estimated interactions. A CHTS method is effected on an experimental space representing the levels and the probabilities for each interaction are adjusted according to results of the CHTS method.

The invention also relates to a system for conducting an experiment. The system comprises a reactor for effecting a CHTS method on an experimental space to produce results and a programmed controller that stores an assigned probability value for estimated positive interactions between levels of factors of the experimental space and adjusts the probabilities for each interaction according to results of the CHTS method.

### BRIEF DESCRIPTION OF DRAWINGS

FIG. 1 is a schematic representation of a system and method for conducting a CHTS experiment.

### DETAILED DESCRIPTION

In one embodiment, the invention provides a method and system to permit a client and an investigator to confer to develop an experiment definition for a CHTS experiment. The method and system can utilize a knowledge matrix as a visual and organizational aid to serve as an adjustable definitional model. The matrix model can include the factors of the experimental space to be investigated. Determination of these factors can require selection of reactant identities and levels and selection of process identities and levels and selection of the degrees of combination. For example, the experimental factors of the catalyst of a carbonylation reaction can be two different metals and a solvent. Levels of one metal may be Fe, Cu, Ni, Pb, and Re, of another metal may be V, W, Ce, La and Sn and of the solvent may be dimethylformamide (DMFA), dimethylacetamide (DMAA), tetrahydrofuran (THF), diglyme (DiGly) or diethylacetamide (DEAA). The model can be set up originally to represent an estimation of factor level interactions. The estimation can take the form of a probability. The experiment can be conducted and a value of the matrix can be adjusted between each iteration of the experiment to represent a probability change dictated by the experiment results.

These and other features will become apparent from the drawings and following detailed discussion, which by way of example without limitation describe preferred embodiments of the present invention.

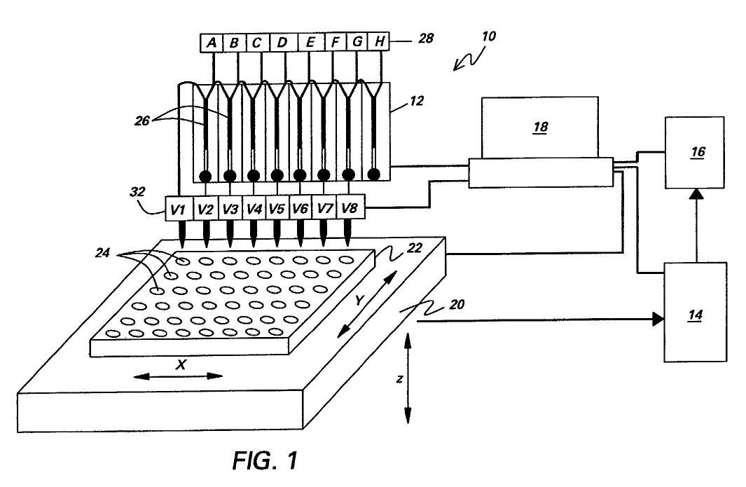

FIG. 1 is a schematic representation of a system 10 and method for conducting a CHTS experiment. FIG. 1 shows system 10 including dispensing assembly 12, reactor 14, detector 16 and controller 18. Further shown, is X-Y-Z robotic positioning stage 20, which supports array plate 22 with wells 24. The dispensing assembly 12 includes a battery of pipettes 26 that are controlled by controller 18. X-Y-Z robotic positioning stage 20 is controlled by controller 18 to position wells 24 of the array plate 22 beneath displacement pipettes 26 for delivery of test solutions from reservoirs 28.

Controller 18 can include a data base repository for storing interaction identifications and probability values input by a client or investigator. The controller 18 also controls aspiration of precursor solution into the battery of pipettes 26 and sequential positioning of the wells 24 of array plate 22 so that a prescribed stoichiometry and/or composition of reactant and/or catalyst can be delivered to the wells 24. By coordinating activation of the pipettes 26 and movement of plate 22 on the robotic X-Y-Z stage 20, a library of materials can be generated in a two-dimensional array for use in the CHTS method. Also, the controller 18 can be used to control sequence of charging of sample to reactor 14 and to control operation of the reactor 14 and the detector 16. Controller 18 can be a computer, processor, microprocessor or the like.

An experimental space is defined according to a design that is embodied as a program resident in controller 18. The design uses input from a client and/or an investigator to define interactions and to assign weights that represent probabilities that the interactions will be positive. Controller 18 translates the defined space into a loading specification for array plate 32. Then controller 18 controls the operation of pipettes 26 and stage 20 according to the specification to deliver reactant and/or catalyst to the wells 34 of plate 22.

Additionally, the controller 18 controls the sequence of charging array plate 22 into the reactor 14, which is synchronized with operation of detector 16. Detector 16 detects products of reaction in the wells 24 of array plate 22 after reaction in reactor 14. Detector 16 can utilize chromatography, infra red spectroscopy, mass spectroscopy, laser mass spectroscopy, microspectroscopy, NMR or the like to determine the constituency of each reaction product. The controller 18 uses data on the sample charged by the pipettes 26 and on the constituency of reaction product for each sample from detector 16 to correlate a detected product with at least one varying parameter of reaction.

As an example, if the method and system of FIG. 1 is applied to study a carbonylation catalyst and/or to determine optimum carbonylation reaction conditions, the detector 16 analyzes the contents of the well for carbonylated product. In this case, the detector 16 can use Raman spectroscopy. The Raman peak is integrated using the analyzer electronics and the resulting data can be stored in the controller 18. Other analytical methods may be used—for example, Infrared spectrometry, mass spectrometry, headspace gas-liquid chromatography and fluorescence detection.

A method of screening complex catalyzed chemical reactions can be conducted in the FIG. 1 system 10. According to the method, a client and an investigator confer to discuss expectations of the experiment to be conducted in the system 10 and the capability of the system to achieve the expectations. The conference can produce a knowledge matrix comprising the experimental space interactions and an assigned weighting to each interaction that represent a first estimate of a probability that the interaction will be a statistically positive interaction, i.e., that the interaction will be a lead. For example, the probabilities can be high, medium and low probabilities. represented respectively by numerical weighting values. “High, medium and low” mean probabilities that are higher, a medium or lower with respect to one another. When three weighting value probabilities are assigned, the values can be in respective ranges of about 0.6 to about 0.99 for high, about 0.2 to about 0.59 for medium and about 0.01 to about 0.19 for low. Desirably, the respective ranges can be about 0.7 to about 0.9, about 0.2 to about 0.5 and about 0.05 to about 0.15. The knowledge matrix is an adjustable definitional model that represents the estimated interactions and assigned or adjusted probabilities. The model can be a visual organizational aid or the model can be a virtual construct resident in a computer database.

Formulations and conditions that represent the interactions are then organized according to an experimental design such as a Latin square design or a full factorial design. Formulations are prepared according to the design. For example, a Latin square design can specify a combination of reactants, catalysts and conditions as a multiphase reactant system. In this procedure, a formulation is prepared that represents a first reactant system that is at least partially embodied in a liquid. Each formulation is loaded as a thin film to a respective well 24 of the array plate 22 and the plate 22 is charged into reactor 14. During the subsequent reaction, the liquid of the first reactant system embodied is contacted with a second reactant system at least partially embodied in a gas. The liquid forms a film having a thickness sufficient to allow the reaction rate of the reaction to be essentially independent of the mass transfer rate of the second reactant system into the liquid.

In one embodiment, the invention is applied to study a process for preparing diaryl carbonates. Diaryl carbonates such as diphenyl carbonate can be prepared by reaction of hydroxyaromatic compounds such as phenol with oxygen and carbon monoxide in the presence of a catalyst composition comprising a Group VIIIB metal such as palladium or a compound thereof, a bromide source such as a quaternary ammonium or hexaalkylguanidinium bromide and a polyaniline in partially oxidized and partially reduced form. The invention can be applied to screen for a catalyst to prepare a diaryl carbonate by carbonylation.

Various methods for the preparation of diaryl carbonates by a carbonylation reaction of hydroxyaromatic compounds with carbon monoxide and oxygen have been disclosed. The carbonylation reaction requires a rather complex catalyst. Reference is made, for example, to Chaudhari et al., U.S. Pat. No. 5,917,077. The catalyst compositions described therein comprise a Group VIIIB metal (i.e., a metal selected from the group consisting of ruthenium, rhodium, palladium, osmium, iridium and platinum) or a complex thereof.

The catalyst material also includes a bromide source. This may be a quaternary ammonium or quaternary phosphonium bromide or a hexaalkylguanidinium bromide. The guanidinium salts are often preferred; they include the ∀, T-bis (pentaalkylguanidinium)alkane salts. Salts in which the alkyl groups contain 2-6 carbon atoms and especially tetra-n-butylammonium bromide and hexaethylguanidinium bromide are particularly preferred.

Other catalytic constituents are necessary in accordance with Chaudhari et al.

The constituents include inorganic cocatalysts, typically complexes of cobalt(II) salts with organic compounds capable of forming complexes, especially pentadentate complexes. Illustrative organic compounds of this type are nitrogen-heterocyclic compounds including pyridines, bipyridines, terpyridines, quinolines, isoquinolines and biquinolines; aliphatic polyamines such as ethylenediamine and tetraalkylethylenediamines; crown ethers; aromatic or aliphatic amine ethers such as cryptanes; and Schiff bases. The especially preferred inorganic cocatalyst in many instances is a cobalt(II) complex with bis-3-(salicylalamino) propylmethylamine.

Organic cocatalysts may be present. These cocatalysts include various terpyridine, phenanthroline, quinoline and isoquinoline compounds including 2,2′:6′,2″-terpyridine, 4-methylthio-2,2′:6′,2″-terpyridine and 2,2′:6′,2″-terpyridine N-oxide,1,10-phenanthroline, 2,4,7,8-tetramethyl-1,10-phenanthroline, 4,7-diphenyl-1,10, phenanthroline and 3,4,7,8-tetramethy-1,10-phenanthroline. The terpyridines and especially 2,2′:6′,2″-terpyridine are preferred.

Another catalyst constituent is a polyaniline in partially oxidized and partially reduced form.

Any hydroxyaromatic compound may be employed. Monohydroxyaromatic compounds, such as phenol, the cresols, the xylenols and p-cumylphenol are preferred with phenol being most preferred. The method may be employed with dihydroxyaromatic compounds such as resorcinol, hydroquinone and 2,2-bis(4-hydroxyphenyl)propane or “bisphenol A,” whereupon the products are polycarbonates.

Other reagents in the carbonylation process are oxygen and carbon monoxide, which react with the phenol to form the desired diaryl carbonate.

The following Example is illustrative and should not be construed as a limitation on the scope of the claims unless a limitation is specifically recited.

### EXAMPLE

This example illustrates an identification of an active and selective catalyst for the production of aromatic carbonates. The procedure includes a combination of a experimental team weighting procedure and a CHTS method to identify a best catalyst from a complex chemical space, where the chemical space is defined as an assemblage of possible experimental conditions defined by a set of variable parameters such as formulation ingredient identity or amount or process parameter such as reaction time, temperature, or pressure.

The chemical space consists of the following TABLE 1 chemical factor levels and TABLE 2 processing factor levels:

TABLE 1

| | Formulation Type Parameter | Formulation Amount |
|---|---|---|
| | Variation | Parameter Variation |
| Precious metal catalyst | Held Constant | Held Constant |
| Primary Transition | Fe, Cu, Ni, Pb, Re (as their | 5,10,20,40 (as molar ratios |
| Metal Cocatalyst (TM) | acetylacetonates) | to precious metal catalyst) |
| Secondary Metal | V, W, Ce, La, Sn (as their | 5,10,20,40 (as molar ratios |
| Cocatalyst (LM) | acetylacetonates) | to precious metal catalyst) |
| Cosolvent (CS) | Dimethylformamide (DMFA), | 50,100,200,400 (as molar |
| | Dimethylacetamide (DMAA), | ratios to precious metal |
| | Diethyl acetamide (DEAA), | catalyst) |
| | Tetrahydrofuran (THF), | |
| | Diglyme (DiGly) | |
| Hydroxyaromatic | Held constant | Sufficient added to achieve |
| compound | | constant sample volume |

Process parameters are shown in TABLE 2:

TABLE 2

| Process Parameter | Parameter Variation |
|---|---|
| Temperature | Constant at 100° C. |
| Pressure | Constant at 1500 psig |

Pre-test estimates of interactions among factor levels are postulated at a meeting between a customer and investigators. The estimates are assigned probability values, which are expressed in the following knowledge matrix TABLE 3. The probabilities are constrained to three possible values, 0.8, 0.3 and 0.1, which express high, medium, and low probabilities. Probabilities of 0.0 and 1.0 are excluded from off-diagonal cells since these probabilities imply complete knowledge. The matrix is symmetrical around the main diagonal, since the probability of A interacting with B is the same as the probability of B interacting with A.

TABLE 3

| | TM | TM | LM | LM | CS | CS |
|---|---|---|---|---|---|---|
| | Type | Amount | Type | Amount | Type | Amount |
| TM Type | 1 | 0.8 | 0.3 | 0.3 | 0.3 | 0.3 |
| TM Amount | 0.8 | 1 | 0.3 | 0.1 | 0.3 | 0.1 |
| LM Type | 0.3 | 0.3 | 1 | 0.8 | 0.3 | 0.1 |
| LM Amount | 0.3 | 0.1 | 0.8 | 1 | 0.1 | 0.1 |
| CS Type | 0.3 | 0.3 | 0.3 | 0.1 | 1 | 0.8 |
| CS Amount | 0.3 | 0.1 | 0.1 | 0.1 | 0.8 | 1 |

The matrix information is loaded into a computer database. The computer defines a full factorial experiment according to two factor interactions between levels as shown in TABLE 4. The computer also controls a dispensing assembly and loading robot to load experimental array trays and a reactor to conduct a CHTS experiment. In the experiment, catalyzed mixtures are made up in phenol solvent using the concentrations of each component as given in the rows of TABLE 4. The total volume of each catalyzed mixture is 1.0 ml. From each mixture, a 25 microliter aliquot is dispensed into a 2 ml reaction vial, forming a film on the bottom. The vials are grouped in array plates by process conditions (as specified in the TABLE 2 Pressure and Temperature columns) and each array plate is loaded into a high pressure autoclave and subjected to the reaction conditions specified. At the end of the reaction time, the reactor is cooled and depressurized and the contents of each vial are analyzed for diphenyl carbonate product using a gas chromatographic method. Performance is expressed numerically as a catalyst turnover number or TON. TON is defined as the number of moles of aromatic carbonate produced per mole of Palladium catalyst charged. This is shown in column TON of TABLE 4.

TABLE 4

| TMType | LMType | CSType | TMAmt | LMAmt | CSAmt | TON |
|---|---|---|---|---|---|---|
| Ni | V | DiGly | 10 | 5 | 200 | 1084 |
| Re | Ce | DEAA | 10 | 10 | 200 | 1394 |
| Ni | La | DMFA | 10 | 40 | 400 | 1221 |
| Ni | Sn | DEAA | 40 | 5 | 50 | 1697 |
| Fe | La | DMAA | 10 | 20 | 200 | 949 |
| Pb | Sn | DEAA | 40 | 10 | 50 | 2317 |
| Fe | Ce | THF | 40 | 20 | 100 | 792 |
| Cu | V | THF | 10 | 10 | 200 | 1054 |
| Cu | Sn | DEAA | 20 | 10 | 100 | 1058 |
| Cu | La | DMAA | 10 | 40 | 50 | 1081 |
| Re | Ce | DMFA | 5 | 40 | 100 | 1058 |
| Re | V | THF | 5 | 5 | 400 | 1074 |
| Cu | W | DMAA | 5 | 40 | 400 | 1125 |
| Cu | Ce | THF | 5 | 10 | 200 | 1111 |
| Ni | La | DMFA | 20 | 5 | 50 | 1358 |
| Fe | Ce | DMFA | 10 | 20 | 50 | 955 |
| Pb | V | DEAA | 10 | 5 | 100 | 1040 |
| Cu | V | DMFA | 20 | 40 | 50 | 1092 |
| Re | V | DMAA | 10 | 10 | 100 | 1080 |
| Re | V | DEAA | 10 | 10 | 50 | 1049 |
| Pb | V | DEAA | 20 | 10 | 400 | 1043 |
| Pb | Ce | THF | 10 | 10 | 100 | 1248 |
| Fe | W | DMAA | 40 | 40 | 50 | 914 |
| Ni | La | DMAA | 5 | 20 | 50 | 1069 |
| Fe | La | DEAA | 5 | 20 | 400 | 1069 |
| Cu | Sn | DiGly | 20 | 40 | 200 | 1114 |
| Cu | W | DMFA | 40 | 10 | 200 | 1105 |
| Pb | Sn | DMAA | 10 | 40 | 50 | 1511 |
| Fe | V | THF | 40 | 10 | 400 | 1067 |
| Re | W | DiGly | 5 | 10 | 400 | 1034 |
| Cu | W | THF | 20 | 10 | 400 | 1041 |
| Pb | La | THF | 10 | 5 | 50 | 1371 |
| Pb | V | DMFA | 20 | 10 | 100 | 1056 |
| Ni | W | THF | 20 | 5 | 200 | 1136 |
| Pb | Sn | DiGly | 10 | 10 | 200 | 1499 |
| Re | W | DMFA | 40 | 5 | 50 | 1535 |
| Ni | Ce | DEAA | 10 | 20 | 200 | 1164 |
| Re | Ce | DEAA | 40 | 20 | 50 | 1959 |
| Re | La | DiGly | 5 | 40 | 200 | 1077 |
| Pb | La | DEAA | 5 | 40 | 50 | 1108 |
| Re | Sn | DiGly | 10 | 40 | 400 | 1660 |
| Ni | V | DMFA | 5 | 5 | 100 | 1083 |
| Re | La | THF | 40 | 5 | 200 | 2396 |
| Re | Sn | DiGly | 20 | 10 | 100 | 2291 |
| Fe | Ce | DMFA | 5 | 40 | 200 | 1029 |
| Re | W | DMAA | 40 | 5 | 400 | 1538 |
| Re | Ce | DiGly | 10 | 20 | 100 | 1417 |
| Ni | Ce | DEAA | 20 | 10 | 400 | 1251 |
| Re | W | DiGly | 20 | 5 | 100 | 1376 |
| Pb | W | THF | 5 | 20 | 50 | 1058 |
| Ni | Ce | THF | 10 | 5 | 100 | 1236 |
| Cu | V | THF | 20 | 40 | 100 | 1078 |
| Fe | Sn | DEAA | 10 | 10 | 400 | 837 |
| Fe | La | DMAA | 20 | 20 | 50 | 805 |
| Re | V | THF | 40 | 20 | 50 | 1076 |
| Pb | W | DiGly | 10 | 20 | 100 | 1194 |
| Fe | W | DEAA | 10 | 40 | 200 | 1017 |
| Fe | Sn | DiGly | 10 | 5 | 50 | 857 |
| Ni | V | DiGly | 20 | 20 | 100 | 1065 |
| Ni | Sn | DMAA | 40 | 40 | 400 | 1645 |
| Re | Sn | THF | 40 | 10 | 100 | 2878 |
| Ni | W | DiGly | 40 | 40 | 100 | 1173 |
| Pb | Sn | DEAA | 5 | 5 | 400 | 1080 |
| Cu | Ce | THF | 40 | 5 | 50 | 1038 |
| Ni | W | DiGly | 20 | 10 | 200 | 1215 |
| Ni | Ce | DEAA | 20 | 5 | 200 | 1275 |
| Re | V | DiGly | 20 | 5 | 50 | 1085 |
| Cu | V | DiGly | 10 | 20 | 400 | 1046 |
| Cu | Sn | DMFA | 10 | 5 | 400 | 1093 |
| Ni | Ce | DiGly | 5 | 5 | 50 | 1069 |
| Pb | V | DiGly | 5 | 40 | 400 | 1039 |
| Fe | W | DEAA | 40 | 5 | 400 | 936 |
| Fe | W | THF | 10 | 10 | 100 | 1043 |
| Re | Ce | DMAA | 20 | 5 | 100 | 1705 |
| Ni | W | DMFA | 20 | 20 | 100 | 1187 |
| Cu | La | DiGly | 5 | 10 | 50 | 1098 |
| Pb | Ce | DMFA | 20 | 40 | 50 | 1458 |
| Pb | V | DMAA | 5 | 10 | 200 | 1113 |
| Pb | V | DMAA | 40 | 20 | 50 | 1072 |
| Ni | Sn | DMAA | 5 | 5 | 100 | 1089 |
| Ni | V | THF | 20 | 40 | 50 | 1092 |
| Re | La | DMFA | 10 | 20 | 200 | 1531 |
| Pb | Ce | DiGly | 5 | 10 | 50 | 1067 |
| Cu | Sn | DMAA | 5 | 20 | 200 | 1034 |
| Fe | Ce | THF | 5 | 40 | 400 | 1105 |
| Pb | V | DMFA | 10 | 40 | 200 | 1110 |
| Re | Sn | DMFA | 5 | 10 | 50 | 1078 |
| Pb | V | THF | 5 | 20 | 200 | 1136 |
| Ni | La | DEAA | 10 | 5 | 100 | 1256 |
| Fe | Sn | THF | 5 | 5 | 200 | 1056 |
| Pb | La | DMAA | 5 | 40 | 100 | 1069 |
| Cu | Ce | DiGly | 20 | 5 | 400 | 1110 |
| Ni | W | DEAA | 5 | 40 | 400 | 1082 |
| Pb | La | DiGly | 5 | 5 | 100 | 1068 |
| Pb | Sn | THF | 20 | 40 | 400 | 1851 |
| Cu | La | DMFA | 10 | 10 | 100 | 1078 |
| Re | Sn | DiGly | 5 | 20 | 50 | 1118 |
| Re | W | THF | 10 | 40 | 50 | 1252 |
| Pb | W | DEAA | 5 | 10 | 200 | 1040 |
| Cu | V | DEAA | 10 | 5 | 50 | 1088 |
| Cu | La | DMFA | 40 | 20 | 50 | 1086 |
| Fe | Sn | DMFA | 5 | 40 | 100 | 1073 |
| Pb | La | DMFA | 40 | 20 | 400 | 1926 |
| Cu | W | THF | 5 | 5 | 100 | 1085 |
| Fe | V | DMFA | 40 | 40 | 400 | 1106 |
| Ni | Ce | THF | 10 | 10 | 50 | 1201 |
| Pb | Ce | DMAA | 20 | 40 | 200 | 1460 |
| Fe | Sn | DEAA | 20 | 5 | 50 | 711 |
| Ni | Sn | THF | 10 | 40 | 200 | 1272 |
| Cu | Ce | DiGly | 10 | 40 | 50 | 1059 |
| Pb | Ce | DMAA | 40 | 5 | 50 | 1718 |
| Fe | V | DiGly | 5 | 10 | 100 | 1060 |
| Pb | W | DMAA | 20 | 10 | 50 | 1292 |
| Re | Ce | DMAA | 5 | 40 | 50 | 1047 |
| Fe | La | DMAA | 20 | 10 | 200 | 792 |
| Re | V | DMFA | 5 | 40 | 50 | 1057 |
| Fe | Sn | THF | 5 | 20 | 400 | 1045 |
| Ni | V | DiGly | 40 | 10 | 50 | 1074 |
| Ni | V | DMAA | 20 | 5 | 400 | 1070 |
| Fe | La | DiGly | 20 | 40 | 100 | 758 |
| Cu | La | DEAA | 5 | 5 | 200 | 1047 |
| Re | La | DiGly | 20 | 20 | 400 | 2009 |
| Pb | Ce | DEAA | 40 | 5 | 100 | 1695 |
| Re | Sn | DMAA | 20 | 20 | 400 | 2255 |
| Pb | La | THF | 20 | 10 | 200 | 1701 |
| Pb | W | DMAA | 40 | 20 | 200 | 1366 |
| Cu | Sn | THF | 5 | 40 | 50 | 1073 |
| Re | Sn | DEAA | 5 | 40 | 200 | 1090 |
| Pb | La | DEAA | 20 | 20 | 100 | 1677 |
| Pb | W | DiGly | 40 | 5 | 50 | 1421 |
| Fe | La | THF | 10 | 40 | 50 | 945 |
| Fe | Sn | DiGly | 40 | 40 | 400 | 453 |
| Pb | Ce | DEAA | 10 | 40 | 400 | 1303 |
| Cu | Sn | DEAA | 40 | 20 | 400 | 1102 |
| Ni | La | DEAA | 10 | 5 | 100 | 1256 |
| Fe | Sn | THF | 5 | 5 | 200 | 1056 |
| Pb | La | DMAA | 5 | 40 | 100 | 1069 |
| Cu | Ce | DiGly | 20 | 5 | 400 | 1110 |
| Ni | W | DEAA | 5 | 40 | 400 | 1082 |
| Pb | La | DiGly | 5 | 5 | 100 | 1068 |
| Pb | Sn | THF | 20 | 40 | 400 | 1851 |
| Cu | La | DMFA | 10 | 10 | 100 | 1078 |
| Re | Sn | DiGly | 5 | 20 | 50 | 1118 |
| Re | W | THF | 10 | 40 | 50 | 1252 |
| Pb | W | DEAA | 5 | 10 | 200 | 1040 |
| Cu | V | DEAA | 10 | 5 | 50 | 1088 |
| Cu | La | DMFA | 40 | 20 | 50 | 1086 |
| Fe | Sn | DMFA | 5 | 40 | 100 | 1073 |
| Pb | La | DMFA | 40 | 20 | 400 | 1926 |
| Cu | W | THF | 5 | 5 | 100 | 1085 |
| Fe | V | DMFA | 40 | 40 | 400 | 1106 |
| Ni | Ce | THF | 10 | 10 | 50 | 1201 |
| Pb | Ce | DMAA | 20 | 40 | 200 | 1460 |
| Fe | Sn | DEAA | 20 | 5 | 50 | 711 |
| Ni | Sn | THF | 10 | 40 | 200 | 1272 |
| Cu | Ce | DiGly | 10 | 40 | 50 | 1059 |
| Pb | Ce | DMAA | 40 | 5 | 50 | 1718 |
| Fe | V | DiGly | 5 | 10 | 100 | 1060 |
| Pb | W | DMAA | 20 | 10 | 50 | 1292 |
| Re | Ce | DMAA | 5 | 40 | 50 | 1047 |
| Fe | La | DMAA | 20 | 10 | 200 | 792 |
| Re | V | DMFA | 5 | 40 | 50 | 1057 |
| Fe | Sn | THF | 5 | 20 | 400 | 1045 |
| Ni | V | DiGly | 40 | 10 | 50 | 1074 |
| Ni | V | DMAA | 20 | 5 | 400 | 1070 |
| Fe | La | DiGly | 20 | 40 | 100 | 758 |
| Cu | La | DEAA | 5 | 5 | 200 | 1047 |
| Re | La | DiGly | 20 | 20 | 400 | 2009 |
| Pb | Ce | DEAA | 40 | 5 | 100 | 1695 |
| Re | Sn | DMAA | 20 | 20 | 400 | 2255 |
| Pb | La | THF | 20 | 10 | 200 | 1701 |
| Pb | W | DMAA | 40 | 20 | 200 | 1366 |
| Cu | Sn | THF | 5 | 40 | 50 | 1073 |
| Re | Sn | DEAA | 5 | 40 | 200 | 1090 |
| Pb | La | DEAA | 20 | 20 | 100 | 1677 |
| Pb | W | DiGly | 40 | 5 | 50 | 1421 |
| Fe | La | THF | 10 | 40 | 50 | 945 |
| Fe | Sn | DiGly | 40 | 40 | 400 | 453 |
| Pb | Ce | DEAA | 10 | 40 | 400 | 1303 |
| Cu | Sn | DEAA | 40 | 20 | 400 | 1102 |
| Fe | W | DMFA | 20 | 5 | 400 | 963 |
| Cu | Sn | DiGly | 5 | 5 | 100 | 1089 |
| Cu | La | THF | 40 | 40 | 50 | 1059 |
| Fe | La | DiGly | 10 | 5 | 400 | 902 |
| Re | Sn | DMFA | 40 | 40 | 400 | 2853 |
| Re | Sn | DiGly | 40 | 40 | 50 | 2870 |
| Pb | W | THF | 40 | 40 | 100 | 1352 |
| Fe | V | DMAA | 5 | 5 | 50 | 1085 |
| Cu | V | DEAA | 5 | 40 | 100 | 1060 |
| Re | Sn | DiGly | 40 | 5 | 400 | 2917 |
| Pb | Sn | DiGly | 40 | 20 | 100 | 2301 |
| Fe | Ce | THF | 20 | 10 | 50 | 868 |
| Fe | Ce | DEAA | 5 | 10 | 100 | 1071 |
| Re | Ce | DiGly | 40 | 40 | 400 | 1987 |
| Re | W | DMFA | 20 | 40 | 200 | 1403 |
| Fe | V | DMAA | 10 | 40 | 100 | 1102 |
| Cu | W | THF | 40 | 20 | 200 | 1059 |
| Re | La | DMFA | 20 | 10 | 400 | 1991 |
| Ni | W | DEAA | 40 | 10 | 100 | 1225 |
| Ni | W | DiGly | 40 | 20 | 400 | 1219 |
| Re | La | THF | 20 | 40 | 100 | 1989 |
| Re | La | DEAA | 40 | 10 | 400 | 2390 |
| Ni | Sn | DMFA | 5 | 40 | 200 | 1096 |
| Re | V | DiGly | 40 | 20 | 200 | 1075 |
| Cu | V | DMFA | 5 | 10 | 400 | 1112 |
| Ni | Sn | DMAA | 20 | 10 | 200 | 1470 |
| Ni | Ce | DMFA | 40 | 40 | 50 | 1411 |
| Re | La | DEAA | 5 | 5 | 50 | 1102 |
| Fe | W | DMAA | 5 | 5 | 100 | 1031 |
| Ni | La | THF | 40 | 5 | 400 | 1545 |
| Fe | Sn | DMFA | 40 | 10 | 200 | 432 |
| Pb | La | DMAA | 10 | 10 | 400 | 1324 |
| Re | Sn | DMAA | 10 | 5 | 200 | 1676 |
| Ni | La | DEAA | 20 | 40 | 200 | 1341 |
| Fe | Ce | DiGly | 10 | 10 | 400 | 995 |
| Re | W | DMAA | 5 | 20 | 100 | 1081 |
| Re | Ce | DMFA | 10 | 5 | 400 | 1379 |
| Ni | W | DMFA | 10 | 10 | 400 | 1075 |
| Cu | W | DEAA | 20 | 40 | 50 | 1037 |
| Ni | La | DMAA | 40 | 10 | 100 | 1522 |
| Pb | Ce | DMFA | 5 | 20 | 400 | 1061 |
| Ni | W | DMAA | 10 | 40 | 200 | 1126 |
| Ni | V | DEAA | 5 | 20 | 400 | 1107 |
| Re | Ce | DMAA | 40 | 10 | 200 | 1919 |
| Ni | Sn | DMFA | 20 | 20 | 400 | 1490 |

The results in TABLE 4 are then subjected to an Analysis of Variance (ANOVA) analysis that includes the main effects and all the two-way interactions of the six factors (TM Type, TM Amount, LM type, LM amount, CS Amount, and CS Type). Results of the ANOVA are shown in TABLE 5.

TABLE 5

| Source | DF | Seq SS | Adj SS | Adj MS | F | P | |
|---|---|---|---|---|---|---|---|
| TMType | 4 | 12344279 | 5926470 | 1481617 | 119.24 | 0.000 | |
| LMType | 4 | 3400185 | 1381835 | 345459 | 27.8 | 0.000 | |
| TMType*LMType | 16 | 5223338 | 2724490 | 170281 | 13.7 | 0.000 | ** |
| CSType | 4 | 171937 | 76127 | 19032 | 1.53 | 0.231 | |
| TMType*CSType | 16 | 788408 | 436537 | 27284 | 2.2 | 0.049 | |
| TMAmount | 3 | 3283677 | 1543785 | 514625 | 41.42 | 0.000 | |
| TMType*TMAmount | 12 | 6432183 | 2597860 | 216488 | 17.42 | 0.000 | ** |
| LMAmount | 3 | 77667 | 6773 | 2258 | 018 | 0.908 | |
| TMType*LMAmount | 12 | 331369 | 195394 | 16283 | 1.31 | 0.287 | |
| CSAmount | 3 | 98658 | 3220 | 1073 | 0.03 | 0.967 | |
| TMType*CSAmount | 12 | 468170 | 284193 | 23683 | 1.91 | 0.098 | |
| LMType*CSType | 16 | 216050 | 364113 | 22757 | 1.83 | 0.100 | |
| LMType*TMAmount | 12 | 1325612 | 966688 | 80557 | 6.48 | 0.000 | ** |
| LMType*LMAmount | 12 | 193246 | 375448 | 31287 | 2.52 | 0.033 | |
| LMType*CSAmount | 12 | 144330 | 211215 | 17601 | 1.42 | 0.237 | |
| CSType*TMAmount | 12 | 143455 | 162020 | 13502 | 1.09 | 0.420 | |
| CSType*LMAmount | 12 | 531604 | 242598 | 20217 | 1.63 | 0.162 | |
| CSType*CSAmount | 12 | 144681 | 174047 | 14504 | 1.17 | 0.367 | |
| TMAmount*LMAmount | 9 | 136750 | 151726 | 16858 | 1.36 | 0.271 | |
| TMAmount*CSAmount | 9 | 140146 | 109713 | 12190 | 0.98 | 0.484 | |
| LMAmount*CSAmount | 9 | 387333 | 387333 | 43037 | 3.46 | 0.010 | * |
| Error | 20 | 248520 | 248520 | 12426 | | | |
| Total | 224 | 36231597 | | | | | |

The client and the investigator observe the rows of TABLE 5 that contain interactions. In the TABLE 5, only three of the interactions, marked **, show very strong evidence of statistical significance (P<0.001), and one, marked *, shows moderately strong evidence (P<0.02). Two show weak evidence (P˜0.05). The rest show no evidence of interaction. The client and the investigator then adjust the weighted probabilities in the computer matrix according to the observed statistically significant results. The probabilities are increased for all the strong interactions and decreased for weak interactions. The following algorithm is used as illustrated in TABLE 6: (1) Very strong interaction: increase the matrix amount by half a distance to 1.0. (2) Moderately strong interaction: increase by 0.25 the distance to 1.0. (3) Weak evidence: no change. (4) No evidence: decrease by half the distance to zero.

TABLE 6

| | | TM | | LM | | CS |
|---|---|---|---|---|---|---|
| | TM type | Amount | LM type | Amount | CS Type | Amount |
| TM type | 1 | 0.8 + .1 | 0.3 + .35 | 0.3 − .15 | 0.3 | 0.3 − .15 |
| TM Amount | 0.8 + .1 | 1 | 0.3 + .35 | 0.1 − .05 | 0.3 − .15 | 0.1 − .05 |
| LM type | 0.3 + .35 | 0.3 + .35 | 1 | 0.8 | 0.3 − .15 | 0.1 − .05 |
| LM Amount | 0.3 − .15 | 0.1 − .05 | 0.8 | 1 | 0.1 − .05 | 0.1 + .225 |
| CS Type | 0.3 | 0.3 − .15 | 0.3 − .15 | 0.1 − .05 | 1 | 0.8 − 0.4 |
| CS Amount | 0.3 − .15 | 0.1 − .05 | 0.1 − .05 | 0.1 + .225 | 0.8 − 0.4 | 1 |

The revisions shown to TABLE 6, result in TABLE 7.

TABLE 7

| | | TM | | LM | | CS |
|---|---|---|---|---|---|---|
| | TM type | Amount | LM type | Amount | CS Type | Amount |
| TM type | 1 | .9 | .65 | .15 | 0.3 | .15 |
| TM Amount | .9 | 1 | .65 | .05 | .15 | .05 |
| LM type | .65 | .65 | 1 | .8 | .15 | .05 |
| LM Amount | .15 | .05 | .8 | 1 | .05 | .325 |
| CS Type | 0.3 | .16 | .15 | .05 | 1 | .4 |
| CS Amount | .15 | .05 | .05 | .325 | .4 | 1 |

A full factorial experiment is organized and run according to the strongest interactions on the TM Type/TM Amount/LM Type variables (5×4×5=100 runs, fully replicated to 200 runs). Results are shown in TABLE 8.

TABLE 8

| | | | TM | LM | | |
|---|---|---|---|---|---|---|
| TMType | LMType | CSType | Amount | Amount | CS Amount | TON |
| Fe | V | DMAA | 5 | 10 | 100 | 1138 |
| Fe | W | DMAA | 5 | 10 | 100 | 1137 |
| Fe | Ce | DMAA | 5 | 10 | 100 | 1357 |
| Fe | La | DMAA | 5 | 10 | 100 | 1424 |
| Fe | Sn | DMAA | 5 | 10 | 100 | 1605 |
| Cu | V | DMAA | 5 | 10 | 100 | 1000 |
| Cu | W | DMAA | 5 | 10 | 100 | 1040 |
| Cu | Ce | DMAA | 5 | 10 | 100 | 1159 |
| Cu | La | DMAA | 5 | 10 | 100 | 1176 |
| Cu | Sn | DMAA | 5 | 10 | 100 | 1048 |
| Ni | V | DMAA | 5 | 10 | 100 | 884 |
| Ni | W | DMAA | 5 | 10 | 100 | 896 |
| Ni | Ce | DMAA | 5 | 10 | 100 | 905 |
| Ni | La | DMAA | 5 | 10 | 100 | 848 |
| Ni | Sn | DMAA | 5 | 10 | 100 | 972 |
| Pb | V | DMAA | 5 | 10 | 100 | 743 |
| Pb | W | DMAA | 5 | 10 | 100 | 965 |
| Pb | Ce | DMAA | 5 | 10 | 100 | 585 |
| Pb | La | DMAA | 5 | 10 | 100 | 709 |
| Pb | Sn | DMAA | 5 | 10 | 100 | 129 |
| Re | V | DMAA | 5 | 10 | 100 | 549 |
| Re | W | DMAA | 5 | 10 | 100 | 767 |
| Re | Ce | DMAA | 5 | 10 | 100 | 491 |
| Re | La | DMAA | 5 | 10 | 100 | 726 |
| Re | Sn | DMAA | 5 | 10 | 100 | 511 |
| Fe | V | DMAA | 10 | 10 | 100 | 1002 |
| Fe | W | DMAA | 10 | 10 | 100 | 1038 |
| Fe | Ce | DMAA | 10 | 10 | 100 | 1124 |
| Fe | La | DMAA | 10 | 10 | 100 | 1211 |
| Fe | Sn | DMAA | 10 | 10 | 100 | 1388 |
| Cu | V | DMAA | 10 | 10 | 100 | 1000 |
| Cu | W | DMAA | 10 | 10 | 100 | 1069 |
| Cu | Ce | DMAA | 10 | 10 | 100 | 1064 |
| Cu | La | DMAA | 10 | 10 | 100 | 1278 |
| Cu | Sn | DMAA | 10 | 10 | 100 | 1269 |
| Ni | V | DMAA | 10 | 10 | 100 | 1061 |
| Ni | W | DMAA | 10 | 10 | 100 | 1136 |
| Ni | Ce | DMAA | 10 | 10 | 100 | 977 |
| Ni | La | DMAA | 10 | 10 | 100 | 1001 |
| Ni | Sn | DMAA | 10 | 10 | 100 | 1487 |
| Pb | V | DMAA | 10 | 10 | 100 | 1048 |
| Pb | W | DMAA | 10 | 10 | 100 | 1188 |
| Pb | Ce | DMAA | 10 | 10 | 100 | 1333 |
| Pb | La | DMAA | 10 | 10 | 100 | 907 |
| Pb | Sn | DMAA | 10 | 10 | 100 | 1155 |
| Re | V | DMAA | 10 | 10 | 100 | 1028 |
| Re | W | DMAA | 10 | 10 | 100 | 839 |
| Re | Ce | DMAA | 10 | 10 | 100 | 834 |
| Re | La | DMAA | 10 | 10 | 100 | 1308 |
| Re | Sn | DMAA | 10 | 10 | 100 | 1203 |
| Fe | V | DMAA | 20 | 10 | 100 | 879 |
| Fe | W | DMAA | 20 | 10 | 100 | 877 |
| Fe | Ce | DMAA | 20 | 10 | 100 | 888 |
| Fe | La | DMAA | 20 | 10 | 100 | 983 |
| Fe | Sn | DMAA | 20 | 10 | 100 | 759 |
| Cu | V | DMAA | 20 | 10 | 100 | 1000 |
| Cu | W | DMAA | 20 | 10 | 100 | 1016 |
| Cu | Ce | DMAA | 20 | 10 | 100 | 1146 |
| Cu | La | DMAA | 20 | 10 | 100 | 1236 |
| Cu | Sn | DMAA | 20 | 10 | 100 | 1205 |
| Ni | V | DMAA | 20 | 10 | 100 | 1149 |
| Ni | W | DMAA | 20 | 10 | 100 | 1062 |
| Ni | Ce | DMAA | 20 | 10 | 100 | 1289 |
| Ni | La | DMAA | 20 | 10 | 100 | 1374 |
| Ni | Sn | DMAA | 20 | 10 | 100 | 1668 |
| Pb | V | DMAA | 20 | 10 | 100 | 1126 |
| Pb | W | DMAA | 20 | 10 | 100 | 1449 |
| Pb | Ce | DMAA | 20 | 10 | 100 | 1476 |
| Pb | La | DMAA | 20 | 10 | 100 | 1592 |
| Pb | Sn | DMAA | 20 | 10 | 100 | 1828 |
| Re | V | DMAA | 20 | 10 | 100 | 1136 |
| Re | W | DMAA | 20 | 10 | 100 | 1728 |
| Re | Ce | DMAA | 20 | 10 | 100 | 1481 |
| Re | La | DMAA | 20 | 10 | 100 | 2336 |
| Re | Sn | DMAA | 20 | 10 | 100 | 1928 |
| Fe | V | DMAA | 40 | 10 | 100 | 765 |
| Fe | W | DMAA | 40 | 10 | 100 | 741 |
| Fe | Ce | DMAA | 40 | 10 | 100 | 715 |
| Fe | La | DMAA | 40 | 10 | 100 | 475 |
| Fe | Sn | DMAA | 40 | 10 | 100 | 590 |
| Cu | V | DMAA | 40 | 10 | 100 | 1000 |
| Cu | W | DMAA | 40 | 10 | 100 | 1061 |
| Cu | Ce | DMAA | 40 | 10 | 100 | 1085 |
| Cu | La | DMAA | 40 | 10 | 100 | 1181 |
| Cu | Sn | DMAA | 40 | 10 | 100 | 1153 |
| Ni | V | DMAA | 40 | 10 | 100 | 1198 |
| Ni | W | DMAA | 40 | 10 | 100 | 1367 |
| Ni | Ce | DMAA | 40 | 10 | 100 | 1514 |
| Ni | La | DMAA | 40 | 10 | 100 | 1754 |
| Ni | Sn | DMAA | 40 | 10 | 100 | 1913 |
| Pb | V | DMAA | 40 | 10 | 100 | 1477 |
| Pb | W | DMAA | 40 | 10 | 100 | 1593 |
| Pb | Ce | DMAA | 40 | 10 | 100 | 1980 |
| Pb | La | DMAA | 40 | 10 | 100 | 2059 |
| Pb | Sn | DMAA | 40 | 10 | 100 | 2252 |
| Re | V | DMAA | 40 | 10 | 100 | 1745 |
| Re | W | DMAA | 40 | 10 | 100 | 1906 |
| Re | Ce | DMAA | 40 | 10 | 100 | 2697 |
| Re | La | DMAA | 40 | 10 | 100 | 2606 |
| Re | Sn | DMAA | 40 | 10 | 100 | 3245 |
| Fe | V | DMAA | 5 | 10 | 100 | 1149 |
| Fe | W | DMAA | 5 | 10 | 100 | 1257 |
| Fe | Ce | DMAA | 5 | 10 | 100 | 1311 |
| Fe | La | DMAA | 5 | 10 | 100 | 1435 |
| Fe | Sn | DMAA | 5 | 10 | 100 | 1524 |
| Cu | V | DMAA | 5 | 10 | 100 | 1000 |
| Cu | W | DMAA | 5 | 10 | 100 | 1032 |
| Cu | Ce | DMAA | 5 | 10 | 100 | 1109 |
| Cu | La | DMAA | 5 | 10 | 100 | 1077 |
| Cu | Sn | DMAA | 5 | 10 | 100 | 1301 |
| Ni | V | DMAA | 5 | 10 | 100 | 853 |
| Ni | W | DMAA | 5 | 10 | 100 | 910 |
| Ni | Ce | DMAA | 5 | 10 | 100 | 863 |
| Ni | La | DMAA | 5 | 10 | 100 | 971 |
| Ni | Sn | DMAA | 5 | 10 | 100 | 799 |
| Pb | V | DMAA | 5 | 10 | 100 | 802 |
| Pb | W | DMAA | 5 | 10 | 100 | 828 |
| Pb | Ce | DMAA | 5 | 10 | 100 | 913 |
| Pb | La | DMAA | 5 | 10 | 100 | 529 |
| Pb | Sn | DMAA | 5 | 10 | 100 | 496 |
| Re | V | DMAA | 5 | 10 | 100 | 691 |
| Re | W | DMAA | 5 | 10 | 100 | 395 |
| Re | Ce | DMAA | 5 | 10 | 100 | 372 |
| Re | La | DMAA | 5 | 10 | 100 | 455 |
| Re | Sn | DMAA | 5 | 10 | 100 | 226 |
| Fe | V | DMAA | 10 | 10 | 100 | 912 |
| Fe | W | DMAA | 10 | 10 | 100 | 1060 |
| Fe | Ce | DMAA | 10 | 10 | 100 | 1104 |
| Fe | La | DMAA | 10 | 10 | 100 | 1009 |
| Fe | Sn | DMAA | 10 | 10 | 100 | 1091 |
| Cu | V | DMAA | 10 | 10 | 100 | 1000 |
| Cu | W | DMAA | 10 | 10 | 100 | 1084 |
| Cu | Ce | DMAA | 10 | 10 | 100 | 1087 |
| Cu | La | DMAA | 10 | 10 | 100 | 1246 |
| Cu | Sn | DMAA | 10 | 10 | 100 | 1261 |
| Ni | V | DMAA | 10 | 10 | 100 | 983 |
| Ni | W | DMAA | 10 | 10 | 100 | 1035 |
| Ni | Ce | DMAA | 10 | 10 | 100 | 1238 |
| Ni | La | DMAA | 10 | 10 | 100 | 1119 |
| Ni | Sn | DMAA | 10 | 10 | 100 | 1188 |
| Pb | V | DMAA | 10 | 10 | 100 | 1210 |
| Pb | W | DMAA | 10 | 10 | 100 | 965 |
| Pb | Ce | DMAA | 10 | 10 | 100 | 1480 |
| Pb | La | DMAA | 10 | 10 | 100 | 1038 |
| Pb | Sn | DMAA | 10 | 10 | 100 | 1182 |
| Re | V | DMAA | 10 | 10 | 100 | 1016 |
| Re | W | DMAA | 10 | 10 | 100 | 979 |
| Re | Ce | DMAA | 10 | 10 | 100 | 828 |
| Re | La | DMAA | 10 | 10 | 100 | 1204 |
| Re | Sn | DMAA | 10 | 10 | 100 | 1313 |
| Fe | V | DMAA | 20 | 10 | 100 | 874 |
| Fe | W | DMAA | 20 | 10 | 100 | 923 |
| Fe | Ce | DMAA | 20 | 10 | 100 | 840 |
| Fe | La | DMAA | 20 | 10 | 100 | 1017 |
| Fe | Sn | DMAA | 20 | 10 | 100 | 700 |
| Cu | V | DMAA | 20 | 10 | 100 | 1000 |
| Cu | W | DMAA | 20 | 10 | 100 | 1046 |
| Cu | Ce | DMAA | 20 | 10 | 100 | 1097 |
| Cu | La | DMAA | 20 | 10 | 100 | 1172 |
| Cu | Sn | DMAA | 20 | 10 | 100 | 1226 |
| Ni | V | DMAA | 20 | 10 | 100 | 1106 |
| Ni | W | DMAA | 20 | 10 | 100 | 1249 |
| Ni | Ce | DMAA | 20 | 10 | 100 | 1201 |
| Ni | La | DMAA | 20 | 10 | 100 | 1331 |
| Ni | Sn | DMAA | 20 | 10 | 100 | 1302 |
| Pb | V | DMAA | 20 | 10 | 100 | 1362 |
| Pb | W | DMAA | 20 | 10 | 100 | 1308 |
| Pb | Ce | DMAA | 20 | 10 | 100 | 1665 |
| Pb | La | DMAA | 20 | 10 | 100 | 1558 |
| Pb | Sn | DMAA | 20 | 10 | 100 | 1942 |
| Re | V | DMAA | 20 | 10 | 100 | 1390 |
| Re | W | DMAA | 20 | 10 | 100 | 1629 |
| Re | Ce | DMAA | 20 | 10 | 100 | 1731 |
| Re | La | DMAA | 20 | 10 | 100 | 2401 |
| Re | Sn | DMAA | 20 | 10 | 100 | 2327 |
| Fe | V | DMAA | 40 | 10 | 100 | 748 |
| Fe | W | DMAA | 40 | 10 | 100 | 674 |
| Fe | Ce | DMAA | 40 | 10 | 100 | 714 |
| Fe | La | DMAA | 40 | 10 | 100 | 691 |
| Fe | Sn | DMAA | 40 | 10 | 100 | 610 |
| Cu | V | DMAA | 40 | 10 | 100 | 1000 |
| Cu | W | DMAA | 40 | 10 | 100 | 1028 |
| Cu | Ce | DMAA | 40 | 10 | 100 | 1012 |
| Cu | La | DMAA | 40 | 10 | 100 | 1227 |
| Cu | Sn | DMAA | 40 | 10 | 100 | 1251 |
| Ni | V | DMAA | 40 | 10 | 100 | 1258 |
| Ni | W | DMAA | 40 | 10 | 100 | 1351 |
| Ni | Ce | DMAA | 40 | 10 | 100 | 1568 |
| Ni | La | DMAA | 40 | 10 | 100 | 1576 |
| Ni | Sn | DMAA | 40 | 10 | 100 | 1663 |
| Pb | V | DMAA | 40 | 10 | 100 | 1437 |
| Pb | W | DMAA | 40 | 10 | 100 | 1786 |
| Pb | Ce | DMAA | 40 | 10 | 100 | 1933 |
| Pb | La | DMAA | 40 | 10 | 100 | 2476 |
| Pb | Sn | DMAA | 40 | 10 | 100 | 2126 |
| Re | V | DMAA | 40 | 10 | 100 | 1447 |
| Re | W | DMAA | 40 | 10 | 100 | 1709 |
| Re | Ce | DMAA | 40 | 10 | 100 | 2329 |
| Re | La | DMAA | 40 | 10 | 100 | 3067 |
| Re | Sn | DMAA | 40 | 10 | 100 | 2904 |

An ANOVA analysis of variance of the TABLE 8 data is illustrated in TABLE 9.

TABLE 9

| Source | DF | Seq SS | Adj SS | Adj MS | F | P |
|---|---|---|---|---|---|---|
| TMType | 4 | 4777245 | 4777246 | 1194311 | 83.10 | 0 |
| LMType | 4 | 2432949 | 2432949 | 608237 | 42.32 | 0 |
| TMAmount | 3 | 10451748 | 10451748 | 3483916 | 242.42 | 0 |
| TMType*LMType | 16 | 1330652 | 1330642 | 83166 | 5.79 | 0 |
| TMType*TMAmount | 12 | 22425009 | 22425009 | 1868751 | 130.03 | 0 |
| LMType*TMAmount | 12 | 1489975 | 1489975 | 124186 | 8.64 | 0 |
| TMType*LMType*TMAmount | 48 | 3450829 | 3450829 | 71892 | 5.00 | 0 |
| Error | 100 | 1437139 | 1437139 | 14371 | | |
| Total | 199 | 47795548 | | | | |

The ANOVA analysis detects a statistically significant 3-way interaction, which is a lead to high value formulations with high levels (TMA=40) of Re in the presence of La or Sn.

While preferred embodiments of the invention have been described, the present invention is capable of variation and modification and therefore should not be limited to the precise details of the Examples. The invention includes changes and alterations that fall within the purview of the following claims.

1. A method to conduct an experiment, comprising steps of:
selecting factors for the experiment;
estimating interactions among levels of the factors assigning a probability value of positive interactions for each of the estimated interactions;
effecting a combinatorial high throughput screening (CHTS) method on an experimental space representing the levels; and
adjusting the probabilities for each interaction according to results of the CHTS method.

2. The method of claim 1, comprising assigning a high probability value, medium probability value or low probability value of each positive interaction for each of the estimated interactions is assigned by a client or investigator.

3. The method of claim 1, wherein a high probability value, medium probability value or low probability value of each positive interaction for each of the estimated interactions.

4. The method of claim 1, wherein an investigator and a client who benefits from results from the CHTS experiment in concert determine a probability value to be assigned.

5. The method of claim 1, comprising assigning values to represent a high probability value, medium probability value and low probability value of each positive interaction for each of the estimated interactions.

6. The method of claim 1, comprising assigning 0.6 to about 0.99 value as a high probability value, about 0.2 to about 0.59 value as a medium probability value and about 0.01 to about 0.19 value as a low probability value.

7. The method of claim 1, comprising assigning 0.7 to about 0.9 value as a high probability value, about 0.2 to about 0.5 value as a medium probability value and about 0.05 to about 0.15 value as a low probability value.

8. The method of claim 1, further comprising repeating a CHTS method step and an adjusting probabilities step until a best set of levels is selected.

9. The method of claim 1, comprising constructing an adjustable definitional model to represent the estimated interactions and assigned probabilities.

10. The method of claim 1, wherein the model is a visual organizational aid.

11. The method of claim 1, wherein the model is a virtual construct resident in a computer database.

12. The method of claim 1, wherein the CHTS method comprises defining a first experimental space by structuring the levels according to a Latin Square strategy.

13 The method of claim 1, wherein the CHTS experiment comprises steps of;
preparing a plurality of reagent compositions;
formulating a combinatorial library of reactants from said plurality of reagent compositions;
effecting parallel reaction of the library to produce products; and
evaluating the products to select a lead from the library of reactants.

14. The method of claim 1, wherein conducting the CHTS experiment comprises providing a reactor plate comprising a substrate with an array of reaction cells containing at least one reactant according to an input factor level and reacting the reactant in parallel with other reactants.

15. The method of claim 1, wherein the CHTS comprises effecting parallel chemical reactions of an array of reactants defined as the experimental space.

16. The method of claim 1, wherein the CHTS comprises effecting parallel chemical reactions on a micro scale on reactants defined as the experimental space.

17. The method of claim 1, wherein the CHTS comprises an iteration of steps of simultaneously reacting a multiplicity of tagged reactants and identifying a multiplicity of tagged products of the reaction and evaluating the identified products after completion of a single or repeated iteration.

18. The method of claim 1, wherein the experimental space factors comprise reactants, catalysts and conditions and the CHTS comprises
(A)(a) reacting a reactant selected from the experimental space under a selected set of catalysts or reaction conditions; and (b) evaluating a set of results of the reacting step; and
(B) reiterating step (A) wherein a selected experimental space selected for a step (a) is chosen as a result of an evaluating step (b) of a preceding iteration of step (A).

19. The method of claim 16, wherein the evaluating step (b) comprises identifying relationships between factor levels of the candidate chemical reaction space; and determining the chemical experimental space according to a full factorial design for the next iteration.

20. The method of claim 16, comprising reiterating (A) until a best set of factor levels of the chemical experimental space is selected.

21. The method of claim 1, wherein the factors include a catalyst system comprising a Group VIII B metal.

22. The method of claim 1, wherein the factors include a catalyst system comprising palladium.

23. The method of claim 1, wherein the factors include a catalyst system comprising a halide composition.

24. The method of claim 1, wherein the factors include an inorganic co-catalyst.

25. The method of claim 1, wherein the factors include a catalyst system includes a combination of inorganic co-catalysts.

26. The method of claim 1, wherein the factors comprise a reactant or catalyst at least partially embodied in a liquid and effecting the CHTS method comprises contacting the reactant or catalyst with an additional reactant at least partially embodied in a gas, wherein the liquid forms a film having a thickness sufficient to allow a reaction rate that is essentially independent of a mass transfer rate of additional reactant into the liquid to synthesize products that comprise the results.

27. A system for conducting an experiment, comprising;
a reactor for effecting a CHTS method on an experimental space to produce results; and
a programmed controller that stores an assigned probability value for estimated positive interactions between levels of factors of the experimental space and adjusts the probabilities for each interaction according to results of the CHTS method.

28. The system of claim 27, wherein the assigned probability value is about 0.6 to about 0.99 value as a high probability value, about 0.2 to about 0.59 value as a medium probability value or about 0.01 to about 0.19 value as a low probability value.

29. The system of claim 27, wherein the assigned probability value is about 0.7 to about 0.9 value as a high probability value, about 0.2 to about 0.5 value as a medium probability value or about 0.05 to about 0.15 value as a low probability value.

30. The system of claim 27, wherein said defines a second experimental space according to the adjusted interaction probabilities.

31. The system of claim 27, wherein the controller is a computer, processor or microprocessor.

32. The system of claim 27, further comprising a dispensing assembly to charge factor levels of reactants or catalysts representing the catalyzed chemical experimental space to wells of an array plate for charging to the reactor.

33. The system of claim 27, wherein the dispensing assembly is controlled by the controller to charge factor levels of reactants or catalysts according to the controller defined space.

34. The system of claim 27, further comprising a detector to detect results of the CHTS method effected in the reactor.
